(19) United States
(12) Patent Application Publication
(10) Pub. No.: US 2008/0193915 A1
(43) Pub. Date: Aug. 14, 2008

(54) Isotope Labeled Dinitrophenylhydrazines and Methods of Use

(75) Inventors: Laszlo Prokai, Mansfield, TX (US); Michael J. Forster, Fort Worth, TX (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignees: UNIVERSITY OF FLORIDA RESEARCH FOUNDATION, INC., Gainesville, FL (US); UNIVERSITY OF NORTH TEXAS HEALTH SCIENCE CENTER AT FORT WORTH, Fort Worth, TX (US)

(21) Appl. No.: 11/575,253

(22) PCT Filed: Sep. 29, 2005
(86) PCT No.: PCT/US2005/035131

§ 371 Date: Dec. 4, 2007

Related U.S. Application Data
(60) Provisional application No. 60/614,951, filed on Sep. 29, 2004.

(51) Int. Cl. C12Q 1/70; G01N 33/00; C12Q 1/00; G01N 33/53; C12Q 1/02

(52) U.S. Cl. 435/5; 436/86; 436/63; 436/94; 435/4; 435/29; 435/7.92

(57) ABSTRACT

The subject invention provides novel isotope-labeled dinitrophenylhydrazines (DNPHs) and methods for their use in detecting and/or quantifying carbonyl groups in proteins and other analytes. In particular, the present invention provides novel methods for identifying biomarkers of oxidative stress, which can be used to either forecast or detect diseases and/or conditions associated with oxidative stress. In one embodiment of the invention, isotope-labeled DNPHs are derived from [¹³C₆]chlorobenzene.

### CROSS-REFERENCE TO RELATED APPLICATIONS

This application claims the benefit of U.S. Provisional application No. 60/614,951, filed Sep. 29, 2004.

### GOVERNMENT SUPPORT

The subject matter of this application has been supported in part by U.S. Government Support under National Institutes of Health P01 AG022550. Accordingly, the U.S. Government has certain rights in this invention.

### BACKGROUND OF THE INVENTION

Free radicals are atoms or groups of atoms with an odd (unpaired) number of electrons and can be formed when oxygen interacts with certain molecules. Free radicals are highly reactive, owing to the tendency of electrons to pair. Thus, once formed, free radicals initiate a chain reaction. For example, whenever a free radical reacts with a non-radical, a chain reaction is initiated until two free radicals react and then terminate the propagation with a 2-electron bond (each radical contributing its single unpaired electron).

Free radicals can damage cells when they react with important cellular components such as DNA, or the cell membrane, which can result in compromised cellular function or even cellular death.

Free-radical oxidative damage has been implicated in almost every major chronic disease. For example, studies have shown that aging, many diseases, physical and emotional stress, UV radiation, strenuous exercise, smoking, and diet can increase the production of free radicals (see Riga, S. et al., “Prolongevity medicine: Antagonic-Stress drug in distress, geriatrics, and related diseases. II. Clinical review—2003,” Ann N.Y. Acad Sci., 1019:401-5 (June 2004); Polidori, M. C. et al., “Physical activity and oxidative stress during aging,” Int J Sports Med., 21(3):154-7 (2000); Irie, M. et al., “Depressive state relates to female oxidative DNA damage via neutrophil activation,” Biochem Biophys Res Commun., 311(4):1014-8 (2003); Nishigori, C. et al., “Role of reactive oxygen species in skin carcinogenesis,” Antioxid Redox Signal., 6(3):561-70 (June 2004); van der Vaart, H. et al., “Acute effects of cigarette smoke on inflammation and oxidative stress: a review,” Thorax., 59(8):713-21 (August 2004); and Zhan, C. D. et al., “Superoxide dismutase, catalase and glutathione peroxidase in the spontaneously hypertensive rat kidney: effect of antioxidant-rich diet,” J Hypertens., 22(10):2025-2033 (October 2004)).

Lipids, sugars, DNA and, in particular, proteins that are oxidized cannot perform their normal function and may even become harmful. Many researchers are convinced that the cumulative effects of free radicals also underlie the gradual deterioration that is the hallmark of aging in all individuals, healthy and sick. Determining the identity of proteins susceptible to oxidation in vitro and in vivo can provide a new level of information that may be critical to understanding the specific pathophysiological consequences of oxidative stress-induced damage.

All reactive oxygen species examined thus far, including reactive nitrogen-species, give rise to protein carbonyls. Specifically, reactive oxygen metabolites oxidize certain proteins comprised of amino acids having hydroxyl groups, resulting in the formation of carbonyl groups. Therefore, unlike specific modification products such as nitrotyrosine, protein carbonylation is a broad biomarker for oxidative stress (Berlett and Stadtman, “Protein oxidation in aging, disease, and oxidative stress,” J Biol Chem., 272:20313-20316 (1997)). Protein carbonyls can be formed by a variety of derivative reactions on amino acid residues (lysyl, histidyl, arginyl, prolyl and threonyl) that are susceptible to oxidative modifications (Stadtman, E. R., “Protein oxidation in aging and age-related diseases,” Ann N.Y. Acad Sci., 928:22-38 (2001); Hensley and Floyd, “Reactive oxygen species and protein oxidation in aging: A look back, a look ahead,” Arch Biochem Biophys., 397:377-383 (2002); and Uchida, K., “Histidine and lysine as targets of oxidative modification,” Amino Acids, 25:249-257 (2003)).

Generally, there are three types of amino acid oxidative modifications that can give rise to protein carbonyls: direct attack by reactive oxygen species (ROS), conjugation with lipid peroxidation products, and reaction with reducing-sugars. Protein carbonylation caused by direct ROS oxidation of amino acid residues often involves metal-containing proteins with the generation of hydroxyl radicals that cause site-specific modifications. Glutamic and aminoadipic semialdehydes are the main carbonyl products of metal-catalyzed oxidation of proteins (Requena, J. R. et al., “Glutamic and aminoadipic semialdehydes are the main carbonyl products of metal-catalyzed oxidation of proteins,” Proc Natl Acad Sci. USA, 98:69-74 (2001); Levine and Stadtman, “Oxidative modification of proteins during Aging,” Experimental Gerontology, 36:1495-1502 (2001); and Stadtman and Levine, “Free radical-mediated oxidation of free amino acids and amino acid residues in proteins,” Amino Acids, 25:207-218 (2003)).

Bioactive lipid hydroperoxides generate stable, relatively long-lived, diffusible molecules that are generally considered to be cytotoxic because of their ability to covalently modify, among others, a variety of proteins (Stadtman and Levine, supra. 2003). The widely studied lipid peroxidation products that can conjugate with protein side chains and can be quantified as protein carbonyls are malondialdehyde (MDA) and 4-hydroxynonenal (4-HNE). MDA and 4-HNE often modify lysine and histidine residues and the modification can also be analyzed by antibodies that are raised against them.

Protein modifications by reducing sugars often lead to the formation of advanced glycation end products (AGEs) that also contribute to protein carbonyl content. Antibodies recognizing AGEs are also commercially available for immunochemical detection of AGE formation on cellular proteins. Physiologically and pathologically, all the above described modifications that contribute to protein carbonylation have been documented in aging and in neurodegenerative disorders (Stadtman, supra. 2001; Dalle-Donne, I. et al., “Protein carbonylation in human diseases,” Trends Mol. Medicine, 9:169-176 (2003)).

The classic approach for the detection of protein carbonyl groups involves the reaction of the protein's carbonyl group with 2,4-dinitrophenylhydrazine (DNPH), a carbonyl specific reagent. DNPH-treated proteins can be quantified either spectrophotometrically (such as quantification of resulting hydrazones at 370 nm, see Levine et al., “Carbonyl assays for determination of oxidatively modified proteins,” Methods Enzymol., 233:346-357 (1994)), or immunochemically by the use of anti-DNP antibodies (see Requena et al., “Recent advances in the analysis of oxidized proteins,” Amino Acids, 25:221-226 (2003)). For example, carbonyl groups can be detected by labeling with tritiated borohydride (Levine et al., “Determination of carbonyl content in oxidatively modified proteins,” Methods Enzymol., 186:464-478 (1990)). For carbonyl analysis, high performance liquid chromatography (HPLC) separation is performed followed by spectroscopy at 357 nm. These techniques for detecting protein carbonyl groups, however, would not be able to determine the nature of the carbonylation reactions. In addition, such techniques are sequential specific, labor intensive, and have difficulties in providing accurate relative quantitation of proteins in two separate samples.

Mass spectrometry-based methods have the potential to determine the nature of protein carbonylation, whether it be from HNE, MDA, AGEs or by direct ROS attack. Proteomics activities have been shifting to direct mass spectrometric analysis that combines protein fractionation/purification with (after proteolytic digest) automated peptide MS/MS (see Aebersold and Mann, “Mass spectrometry-based proteomics,” Nature, 422:198-207 (2003)) and, if accurate quantification is desired, stable-isotope tagging of proteins or peptides (see Gygi, S. P. et al., “Quantitative analysis of complex protein mixtures using isotope-coded affinity tags,” Nature Biotech., 17:994-999 (1999); and Gerber, S. A. et al., “Absolute quantification of proteins and phosphoproteins from cell lysates by tandem MS,” Proc Natl Acad Sci USA, 100:6940-6945 (2003)).

Immunochemical techniques have been previously applied to the detection of carbonyl groups in proteins that have been purified and separated by polyacrylamide gel electrophoresis (see Shacter et al., “Differential susceptibility of plasma proteins to oxidative modification: examination by western blot immunoassay,” Free Radical Biol Med., 17:429-437 (1994); and Robinson et al., “Determination of protein carbonyl groups by immunoblotting,” Analyt Biochem., 266:48-57 (1999)). Initial studies identifying oxidized proteins in the brain have used gel electrophoresis with subsequent identification of protein spots by peptide-mass fingerprinting based on in-gel proteolytic digestion followed by MALDI-TOF mass spectrometry (Castegna, A. et al., “Proteomic identification of oxidatively modified proteins in Alzheimer's disease brain. Part I: creatine kinase BB, glutamine synthase, and ubiquitin carboxy-terminal hydrolase L-1,” Free Radic Biol Med., 33:562-571 (2002a); Castegna, A. et al., “Proteomic identification of oxidatively modified proteins in Alzheimer's disease brain. Part II: dihydropyrimidinase-related protein 2, alpha-enolase and heat shock cognate 71,” J Neurochem., 82:1524-1532 (2002b); Butterfield and Castegna, “Proteomic analysis of oxidatively modified proteins in Alzheimer's disease brain: Insights into neurodegeneration,” Cell Mol Biol., 49:747-751 (2003a); and Butterfield and Castegna, “Proteomics for the identification of specifically oxidized proteins in brain: Technology and application to the study of neurodegenerative disorders,” Amino Acids, 25:419-425 (2003b)).

Although such techniques have been conceptually straightforward and useful in identifying several age-associated oxidation-sensitive proteins (see Choi et al., “Proteomic identification of specific oxidized proteins in ApoE-knockout mice: Relevance to Alzheimer's disease,” Free Radical Biol Med., 36:1155-1162 (2004)), they remain technically complicated, labor-intensive and difficult to automate at the interface of gel-electrophoresis and mass spectrometry to afford consistent performance for routine application. Additionally, an inherent limitation of the gel-based approach for protein identification is that it is constrained to the most abundant proteins in the samples (see Tyers and Mann, “From Genomics to proteomics,” Nature, 422:193-197 (2003)) and very likely precludes the detection of oxidation associated with low abundance proteins having important brain functions.

A recent study (see Soreghan, B. A. et al., “High-throughput proteomic-based identification of oxidatively induced protein carbonylation in mouse brain,” Pharmaceut Res., 20:1713-1720 (2003)) has demonstrated the power of affinity purification combined with liquid chromatography electrospray ionisation tandem mass spectrometry (LC/ESI-MS/MS) to address the identification of oxidatively induced protein carbonylation in mouse brain upon aging. Many oxidized proteins not revealed by the 2D-GE/in-gel digest/MALDI-MS approach (low-abundance receptors, mitochondrial proteins involved in glucose and energy metabolism, a series of receptors/phosphatases associated with insulin and IGF metabolism and cell-signaling pathways, etc.) were identified by the techniques. See also Fenaille, F. et al., “Immunoaffinity purification and characterization of 4-hydroxy-2-nonenal- and malondialdehyde-modified peptides by electrospray ionization tandem mass spectrometry,” Anal Chem., 74-6298-6304 (2002)). Unfortunately, current methods for detecting protein carbonyl groups using mass spectrometry-based methods have not been effective because accurate and timely quantification of oxidatively induced protein carbonylation is not amenable by these approaches.

All of the methods for detection, as described above, are limited in their usefulness and applicability due to the low specificity and system-limited nature of the markers used for detection. The present invention, in contrast, provides a highly specific marker for the existence and detection/measurement of proteins susceptible to oxidation, which can provide information that may be critical to understanding the specific pathophysiological consequences of oxidative stress-induced damage.

### BRIEF SUMMARY OF THE SUBJECT INVENTION

The subject invention provides materials and methods for the detection of carbonylated analytes for use in identifying and/or quantifying analytes susceptible to oxidation.

In a preferred embodiment of the invention, proteins that give rise to a reactive carbonyl upon oxidation are identified using the compounds and methods of the invention. As noted above, there appears to be a relationship between the number of protein carbonyl groups and oxidative stress and subsequent diseases or conditions associated with oxidative stress. Accordingly, one object of the invention is the identification and usage of protein carbonyl groups as biomarkers of oxidative stress. The usage of protein carbonyl groups as biomarkers of oxidative stress is particularly advantageous when compared with the measurement of other oxidation products because of the relatively early formation and the relative stability of carbonylated proteins.

The present invention provides novel isotope labeled DNPH for use in assaying protein carbonylation (as the result of oxidation), identifying proteins susceptible to oxidation, and identifying potential biomarkers of oxidative stress and subsequent diseases or conditions associated with oxidative stress. The isotope labeled DNPH of the subject invention is useful in protein derivatization that, when combined with other detection techniques, provides improved peptide quantification measurements as compared to those provided using previously disclosed detection techniques.

In a preferred embodiment, stable-isotope labeled reagents of the invention are used to enable mass spectrometric identification and differential quantification of carbonylated proteins/peptides by the isotope-coded affinity tagging (ICAT) method with, specifically, the DNPH-derivatization providing the affinity tag.

One method of the invention comprises the following steps: (i) protein derivatization by DNPH and stable-isotope labeled DNPH; (ii) gel-based purification and proteolysation of derivatized proteins; (iii) determination of specifically carbonylated proteins. In certain embodiments, purified and separated derivatized peptides/proteins are contacted with anti-DNPH immunosorbent prior to the step of determining specifically carbonylated proteins. By treating expressed proteins with two isotopically variant chemical reagents (also termed isotope-coded affinity tags or ICATs) such as isotope labeled DNPH and non-labeled DNPH, the subject invention enables accurate detection and comparison of protein expression from different sample sources under a range of experimental conditions.

A second contemplated method for detecting oxidatively induced protein carbonylation is also based on ICAT, which comprises the following steps: (i) protein derivatization by DNPH and stable-isotope labeled DNPH of the invention; (ii) gel-free separation and proteolysation of derivatized proteins; (iii) determination of specifically carbonylated proteins. In certain embodiments, purified and separated derivatized peptides/proteins are contacted with anti-DNPH immunosorbent prior to the step of determining specifically carbonylated proteins.

In another aspect, provided are articles of manufacture where the functionality of a method of the invention is embedded on a computer-readable medium, such as, but not limited to, a floppy disk, a hard disk, an optical disk, a magnetic tape, a PROM, an EPROM, CD-ROM, DVD-ROM, or resident in computer or processor memory. The functionality of the method can be embedded on the computer-readable medium in any number of computer readable instructions, or languages such as, for example; FORTRAN, PASCAL, C, C++, BASIC and, assembly language. Further, the computer-readable instructions can, for example, be written in a, script, macro, or functionally embedded in commercially available software, (e.g. EXCEL or VISUAL BASIC).

### BRIEF DESCRIPTION OF THE DRAWINGS

FIGS. 1A and 1B are preparatory schemes for isotope labeled DNPH in accordance with the subject invention.

FIG. 2A illustrate negative-ion atmospheric-pressure chemical ionization mass spectrum of isotope labeled DNPH.

FIG. 2B illustrate negative-ion atmospheric-pressure chemical ionization mass spectrum of non-isotope labeled DNPH.

FIG. 3 illustrates ¹H-NMR spectrum analysis of an isotope labeled DNPH in accordance with the subject invention.

FIG. 4A is a flow diagram of a method for detecting carbonylated proteins in accordance with the subject invention.

FIG. 4B is a flow diagram of another method for detecting carbonylated proteins using isotopically labeled DNPH, in accordance with the subject invention.

FIG. 5 is a preparatory scheme of the formation of a stable DNP hydrazone product as the result of DNPH reaction with a protein in accordance with the subject invention.

### DETAILED DISCLOSURE OF THE SUBJECT INVENTION

The present invention relates to assaying analyte carbonylation and identifying analytes susceptible to oxidation. More particularly, the invention relates to materials and methods useful in detecting carbonylated proteins and/or peptides that can provide information critical to understanding the specific pathophysiological consequences of oxidative stress-induced damage.

The present invention provides novel isotope labeled dinitrophenylhydrazines (DNPH) for use in protein derivatization. As understood by the skilled artisan, any stable isotope can be used in accordance with the subject invention. Contemplated isotopes include ¹³C, ¹⁵N, ¹⁸O, and ²H, all of which can be used to isotopically label any constitutive elements of DNPH. In a preferred embodiment, ¹³C is used to isotopically label DNPH.

In one embodiment of the invention, 2,4-dinitro-[¹³C₆]phenylhydrazine is provided for use in protein derivatization. In another embodiment, 2,4-di-[¹⁵N]nitrophenylhydrazine is provided for use in protein derivatization. In another embodiment, 2,4-di-[¹⁸O]nitrophenylhydrazine is provided for use in protein derivatization. Further embodiments provided by the subject invention can include, but are not limited to, 2,4-dinitrophyenyl[¹⁵N]hydrazine, 2,4-di-[¹⁵N,¹⁸O]nitrophyenyl-hydrazine, and 2,4-di-[¹⁵N,¹⁸O]nitrophyenyl[¹⁵N]hydrazine. Accordingly, the subject invention provides DNPH labeled with multiple isotopes.

In a preferred embodiment, stable-isotope labeled reagents of the invention are used to enable mass spectrometric identification and differential quantification of carbonylated proteins/peptides by an isotope-coded affinity tagging (ICAT) method with, specifically, the isotopically labeled DNPH providing the affinity tag.

The ICAT method, in general, employs chemical reagents termed isotope-coded affinity tags for derivatives of proteins. At least two protein mixtures or samples, which can be from the same source or from different sources, are respectively treated with isotope labeled ICAT reagents, which covalently bond to every carbonyl residue. After the protein mixtures are combined, they are separated and proteolysed to peptides. The ICAT-labeled peptides are isolated and then separated and quantified using known techniques (i.e., mass spectrometry, MALDI-MS and MS/MS, and LC/ESI-MS/MS, etc.). Each pair of ICAT-labeled peptides essentially co-elutes because they are virtually chemically identical. Purified, separated, and digested proteins/peptides will exhibit a mass difference equal to that of the isotope labeled ICAT reagents.

According to the subject invention, the term “sample” refers to a mixture of molecules that include analytes of interest. Samples containing the analyte can be obtained from any source including, but are not limited to, any biological or environmental source. For example, the sample may be a biological material, such as fermentation fluid, soil, water, food, pharmaceutical, organ culture, tissue culture, cell culture, ascites fluid; any plant tissue or extract including root, stem, leaf, or seed, exhaled breath, whole blood, blood plasma, urine, semen, saliva, lymph fluid, meningal fluid, amniotic fluid, glandular fluid, sputum, feces, sweat, mucous, cerebrospinal fluid, and experimentally separated fractions of all of the preceding solutions or mixtures containing homogenized solid material, such as feces, organs, tissues, and biopsy samples.

As used herein, the term “analyte” includes, but is not limited to, the following: organic molecules and inorganic molecules (including synthetic materials, naturally occurring materials, modified naturally occurring materials, polymers, cellulose, nitrocellulose, cellulose acetate, polyvinyl chloride, polyacrylamide, cross-linked dextran molecules, agarose, polystyrene, polyethylene, polypropylene, polymethacrylate, nylon; ceramics, glass metals, magnetite, carbohydrates, and any mixtures or combinations thereof); peptides; proteins; glycoproteins; nucleic acids; lipids; neurotransmitters; hormones; growth factors; antineoplastic agents; cytokines; monokines; lymphokines; enzymes; receptors; DNA; RNA; cells (eucaryotic and procaryotic cells), including all animal and plant cells, stem cells, and blood cells (e.g., reticulocytes, lymphocytes); detectable components of organelles and cells; and microorganisms, such as fungi, viruses, yeast, mycoplasms, bacteria including but not limited to all gram positive and gram negative bacteria, and protozoa.

The term “patient,” as used herein, describes an animal, including mammals, for which biomarkers of oxidative stress can be identified using materials and methods of the subject invention. Mammalian species that benefit from the disclosed methods of the invention include, and are not limited to, apes, chimpanzees, orangutans, humans, monkeys; and domesticated animals such as mice, rats, dogs, cats, guinea pigs, and hamsters.

In accordance with the subject invention, ¹³C, ¹⁵N and/or ¹⁸O are incorporated into DNPH to form a “heavy” ICAT or reagent. DNPH is the “light” reagent. Using both DNPH and isotope labeled DNPH ICAT variants, the subject invention provides materials that are useful in ICAT assay strategy. Specifically, the ICAT variants of the invention result in the co-elution of ICAT-reagent labeled pairs from the reversed-phase column and, thus, provide accurate peptide quantification measurements from two samples, including their relative abundances.

Contemplated methods for detecting protein carbonylation as the result of oxidation are based on an ICAT strategy. One method of the invention comprises the following steps: (i) protein derivatization by DNPH and stable-isotope labeled DNPH; (ii) gel-based purification and proteolysation of derivatized proteins; (iii) determination of specifically carbonylated proteins. In certain embodiments, purified and separated derivatized peptides/proteins are contacted with anti-DNPH immunosorbent prior to the step of determining specifically carbonylated proteins. By treating expressed proteins with two isotopically variant chemical reagents (also termed isotope-coded affinity tags or ICATs) such as isotope labeled DNPH and non-labeled DNPH, the subject invention enables accurate assay and comparison of protein expression from different sample sources under a range of experimental conditions.

In a preferred embodiment, detection of protein carbonylation comprises the following steps: (i) reacting a first sample with DNPH; (ii) reacting a second sample with a stable-isotope labeled DNPH (iso-DNPH); (iii) mixing the samples from steps (i) and (ii) together; (iv) the combined samples of step (iii) are subjected to gel electrophoresis and enzymatic digestion, such as with trypsin, resulting in DNPH-labeled and iso-DNPH-labeled peptides; (v) determining the quantity and sequence of labeled peptides (using known methods such as liquid chromatography/mass spectrometry); and (vi) using the results from step (v) to identify proteins susceptible to oxidation and corresponding diseases.

Preferably, the first and second samples are from the same source. More preferably, the first and second samples comprise proteins that have been oxidized to form carbonyl groups. In certain embodiments, additional samples are reacted with iso-DNPH (different from any already used in step (ii)) and subsequently combined, separated, proteolysed, and analyzed and described above in steps (iv) through (vi). In other embodiments, with a known sequence of a labeled peptide (as provided in step (v)), the identity of the corresponding protein is easily determined by screening peptide, protein, and/or nucleic acid sequence databases. Both the databases and the software to screen are available in the art.

Another contemplated method for detecting oxidatively induced protein carbonylation is also based on ICAT, which comprises the following steps: (i) protein derivatization by DNPH and stable-isotope labeled DNPH of the invention; (ii) gel-free separation and proteolysation of derivatized proteins; (iii) determination of specifically carbonylated proteins. In certain embodiments, purified and separated derivatized peptides/proteins are contacted with anti-DNPH immunosorbent prior to the step of determining specifically carbonylated proteins.

In a preferred embodiment, detection of protein carbonylation comprises the following steps: (i) reacting a first sample with DNPH; (ii) reacting a second sample with a stable-isotope labeled DNPH (iso-DNPH); (iii) mixing the samples from steps (i) and (ii) together; (iv) the combined samples of step (iii) are subjected to enzymatic digestion, such as with trypsin, resulting in DNPH-labeled and iso-DNPH-labeled peptides; (v) determining the quantity and sequence of labeled peptides (using known methods such as liquid chromatography/mass spectrometry); and (vi) using the results from step (v) to identify proteins susceptible to oxidation and corresponding diseases.

Preferably, the first and second samples are from the same source. More preferably, the first and second samples comprise proteins that have been oxidized to form carbonyl groups. In certain embodiments, additional samples are reacted with iso-DNPH (different from any already used in step (ii)) and subsequently combined, separated, proteolysed, and analyzed and described above in steps (iv) through (vi). In other embodiments, with a known sequence of a labeled peptide (as provided in step (v)), the identity of the corresponding protein is easily determined by screening peptide, protein, and/or nucleic acid sequence databases. Both the databases and the software to screen are available in the art.

According to the subject invention, carbonylated proteins can be quantified and/or sequenced by using any known assay techniques including, but not limited to, spectrophotometric assay (such as ultra-violet spectroscopy), enzyme-linked immunosorbent assay (ELISA), one-dimensional or two-dimensional electrophoresis followed by Western blot immunoassay, and other fractionation methods (such as liquid chromatography (LC); two-dimensional liquid chromatography (2D-LC); mass spectrometry (MS); high-performance liquid chromatography (HPLC); HPLC-mass spectrometry (HPLC-MS); electrospray ionization (ESI(−)) and atmospheric pressure chemical ionization (APCI(−)), single ion monitoring (SIM)). Preferred detection techniques for determining specifically carbonylated proteins, in accordance with the subject invention, include LC/MS; HPLC; LC/ESI-MS/MS; MALDI-MS; and MS/MS.

To identify the analyte (such as a protein) susceptible to oxidative stress, the peptide sequences are analyzed and compared against a library of sequences of known proteins. As understood by one skilled in the art, a protein can be identified based on the identification of one or more of its constituting peptides. In one embodiment, a computer algorithm is used to search a protein sequence database to identify the protein(s) associated with the peptide(s) that are quantified and sequenced in accordance with the subject invention.

Protein identification software used in the present invention to compare the experimental mass spectra of the peptides with a database of the peptide masses and the corresponding proteins are available in the art. One such algorithm, ProFound, uses a Bayesian algorithm to search protein or DNA database to identify the optimum match between the experimental data and the protein in the database. ProFound is taught in J. Am. Soc. Mass. Spectrom 10, 91; Patterson S. D., (2000), Am. Physiol. Soc., 59-65; and Yates J R (1998) Electrophoresis, 19, 893. MS/MS spectra may also be analysed by MASCOT (available at Matrix Science Ltd. London).

In some embodiments, where mass spectrometry analysis is used to identify, quantify, and sequence the labeled analytes, a comparison of at least a portion of one or more of the mass spectra results against known (or predicted) mass spectra is used to provide search result dependent data. For example, a peptide mass fingerprinting (PMF) technique can be used to provide putative identifications of analytes in the sample.

As understood by the skilled artisan, various software tools can be used (such as Applied Biosystems 4700 Proteomics Analyzer, Sequest, PeptideProphet, Xpress, ASAPRAtio, Peak Picker, Peak Extraction, Parser, and QuantFixer) to quantify, organize, and identify the peptides and/or proteins in the samples using searchable databases. Several searchable databases are known in the art such as Protein Prospector™ (U. California San Francisco) or Mascot® (Matrix Sciences Ltd.).

In various embodiments, the information obtained from the analysis of the samples containing analytes using the methods described herein are used to identify those analytes susceptible to oxidation and associating at least a portion of this information with a clinical relational or clinical object oriented database. For example, based on the association with clinical information in the relational database or object oriented database, an analyte is characterized as a biomarker for a disease, disorder, or condition.

As noted earlier, there appears to be a relationship between the number of protein carbonyl groups and oxidative stress and subsequent diseases, disorders or conditions associated with oxidative stress. Accordingly, one embodiment of the invention is a method for identifying, and subsequently using, protein carbonyl groups as biomarkers of oxidative stress.

The subject invention contemplates identifying any analyte that gives rise to a reactive carbonyl group upon oxidation. Examples of analytes that can be assayed using the compounds and methods of the invention include, but are not limited to, proteins, lipids, DNA, RNA, eucaryotic and procaryotic cells, including protoplasts; and/or other biological materials such as tissue culture cells, animal cells, animal tissue, blood cells (e.g., reticulocytes, lymphocytes), plant cells, bacteria, yeasts, viruses, mycoplasmas, protozoa, fungi and the like.

As contemplated herein, such biomarkers would be useful in forecasting or detecting diseases, disorders or conditions associated with oxidative stress. For example, the subject invention provides materials and methods for identifying those proteins susceptible to, and thus biomarkers of, oxidative stress that may be used to forecast a pathological situation earlier than the actual manifestation of symptoms. Once a disease, disorder, or condition is forecasted or detected using a biomarker of the invention, appropriate clinical or treatment measures can be taken to prevent and/or treat the disease, disorder or condition.

Examples of diseases, disorders and conditions that can be forecasted or detected in a patient using the materials and methods of the subject invention include, but are not limited to, neurological and neurodegenerative diseases and conditions such as age-associated dementia, Alzheimer's disease, Parkinson's disease, amyotrophic lateral sclerosis (ALS), multiple sclerosis, peripheral neuropathy, shingles, stroke, traumatic injury, and various neurological and other degenerative consequences of neurological and chest surgeries, schizophrenia, epilepsy, Down's Syndrome, and Turner's Syndrome; degenerative conditions associated with AIDS; various bone disorders including osteoporosis, osteomyelitis, ischemic bone disease, fibrous dysplasia, rickets, Cushing's syndrome and osteoarthritis; other types of arthritis and conditions of connective tissue and cartilage degeneration including rheumatoid, psoriatic and infectious arthritis; various infectious diseases; muscle wasting disorders such as muscular dystrophy; skin disorders such as dermatitis, eczema, psoriasis and skin aging; degenerative disorders of the eye including macular degeneration and retinal degeneration; disorders of the ear such as otosclerosis; impaired wound healing; various cardiovascular diseases and conditions including stroke, cardiac ischemia, myocardial infarction, chronic or acute heart failure, cardiac dysrhymias, artrial fibrillation, paroxysmal tachycardia, ventricular fibrillation and congestive heart failure; circulatory disorders including atherosclerosis, arterial sclerosis and peripheral vascular disease, diabetes (Type I or Type II); various diseases of the lung including lung cancer, pneumonia, chronic obstructive lung disease (bronchitis, emphysemia, asthma); disorders of the gastrointestinal tract such as ulcers and hernia; dental conditions such as periodontitis; liver diseases including hepatitis and cirrhosis; pancreatic ailments including acute pancreatitis; kidney diseases such as acute renal failure and glomerulonepritis; and various blood disorders such as vascular amyloidosis, aneurysms, anemia, hemorrhage, sickle cell anemia, autoimmune disease, red blood cell fragmentation syndrome, neutropenia, leukopenia, bone marrow aphasia, pancytopenia, thrombocytopenia, and hemophilia. The preceding list of diseases and conditions which are treatable according to the subject invention is not intended to be exhaustive or limiting but presented as examples of such degenerative diseases and conditions.

In plants all stress phenomena—biotic and abiotic—are accompanied by an increased production of reactive oxygen species (ROS) and this can lead to damage to proteins, lipids and DNA. In green plant cells in the light the chloroplasts and peroxisomes appear to be major sites of ROS production. In contrast, in nongreen plant cells and in green plant cells in darkness the electron transport chain in the mitochondria appears to be the major ROS producer like it is in mammalian cells (see, for example, Møller, I. M., “Plant mitochondria and oxidative stress: electron transport, NADPH turnover, and metabolism of reactive oxygen species,” Annu. Rev. Plant Physiol. Plant Mol. Biol. 52:561-591 (2001); and Foyer, C. H. and Noctor, G., “Oxygen processing in photosynthesis: regulation and signaling,” New Phytol., 146:359-388 (2000)). Accordingly, the subject invention provides materials and methods for identifying those analytes susceptible to, and thus biomarkers of, oxidative stress that may be used to forecast plant pathological situations earlier than the actual manifestation of symptoms. Preferably, proteins in mitochondria that are susceptible to and biomarkers of oxidative stress may be identified using the systems and methods of the subject invention.

Following are examples, which illustrate procedures for practicing the invention. These examples should not be construed as limiting. All percentages are by weight and all solvent mixture proportions are by volume unless otherwise noted.

### EXAMPLE 1

### Synthesis of Stable-Isotope Labeled DNPH

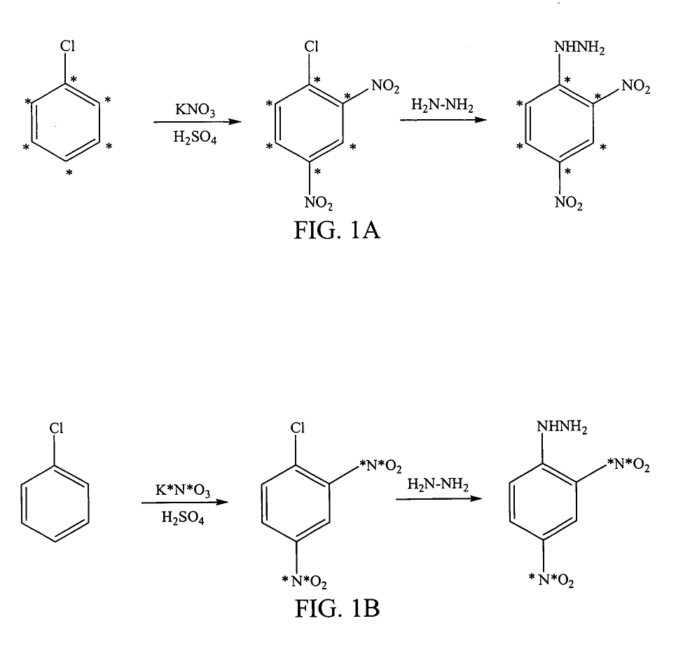

The commercially available [¹³C₆]chlorobenzene and [¹³C₆]bromobenzene (Aldrich, Milwaukee, Wis.) are excellent starting material for the preparation of 2,4-dinitro-[¹³C₆]chlorobenzene and 2,4-dinitro-[¹³C₆]bromobenzene, respectively, as intermediates that can be easily converted to 2,4-dinitro-[¹³C₆]phenylhydrazine at high yield. FIGS. 1A and 1B illustrate the preparation scheme for 2,4-dinitro-[¹³C₆]chlorobenzene as intermediates using KNO₃ (see FIG. 1A) or, alternatively, K¹⁵N¹⁸O₃ (Aldrich, or reagents introducing any suitable combination of appropriate ¹³C-, ¹⁵N- and ¹⁸O-label into the final product, see FIG. 1B). The intermediates are then converted to 2,4-dinitro-[¹³C₆]-phenylhydrazine, where the asterisks indicate the labeled atoms.

To prepare 2,4-dinitro-[¹³C₆]chlorobenzene intermediate, see FIG. 1A, KNO₃ is added to a mixture of 0.7 g (6.2 mmol) [¹³C₆]chlorobenzene in 4 ml carbon tetrachloride and 6.2 ml cc. H₂SO₄, 1.5 g (14.9 mmol) under stirring in 1 h, while the temperature is maintained below 5° C. Then, the solution is stirred at 45° C. for an additional 2.5 h. The mixture is poured onto ice and extracted with 4×10 ml ether, washed with water, and dried over anhydrous sodium sulfate. The solvent is removed in vacuo and the residue is recrystallized form methanol.

The conversion of 2,4-dinitro-[¹³C₆]chlorobenzene to the isotope labeled DNPH of the invention can be performed according to known organic chemistry procedures (Furniss et al., Vogel's Textbook of Practical Organic Chemistry, 5^{th} Ed., Longman, Harlow, UK, pp. 960-961 (1989)). To a solution of 0.5 g (2.5 mmol) of 2,4-dinitro-[¹³C₆]chlorobenzene in 3 mL of rectified spirit, hydrazine (2.5 mmol in 60% aqueous solution) is added and the mixture is refluxed with stirring for an hour. Most of the reaction product separates during the first 10 minutes. After cooling, the product is filtered off, washed with 0.5 ml of warm (60° C.) rectified spirit to remove unchanged chlorodinitrobenzene, and then with 1.0 ml of hot water.

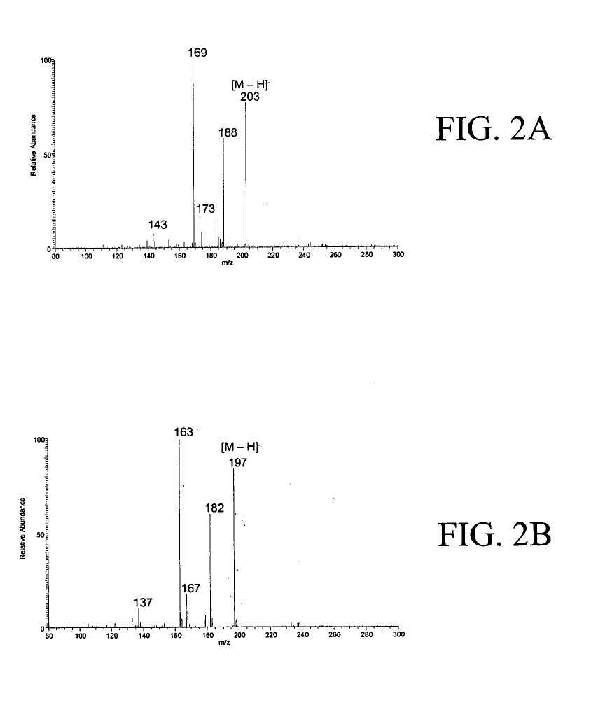

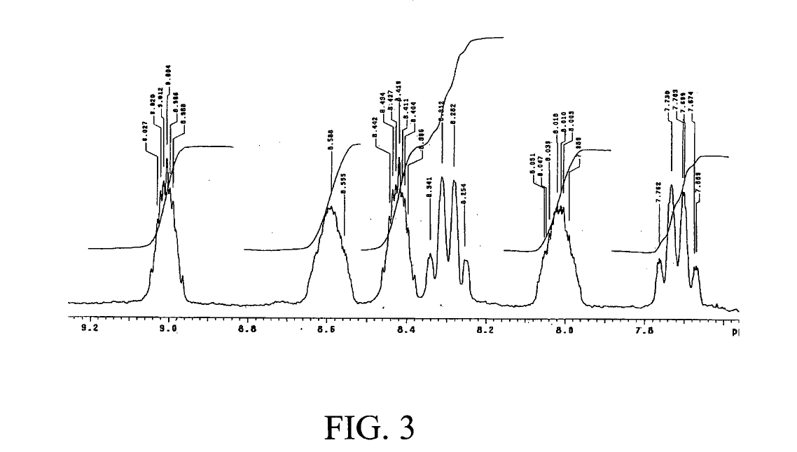

The crude product is recrystallized from 1-butanol or from dioxane. The recrystallized isotope labeled DNPH, as well as the non-labeled DNPH, were characterized by mass spectrometry (see FIGS. 2A and 2B) and NMR (see FIG. 3). FIG. 2A illustrates negative-ion atmospheric pressure chemical ion atmospheric pressure chemical ionization mass spectrum of 2,4-dinitro-[¹³C₆]phenylhydrazine, which shows a +6 u shift in mass-to-charge values, when compared to the non-labeled standard DNPH (Aldrich, Milwaukee, Wis.), as illustrated in FIG. 2B. FIG. 3 illustrates an ¹H-NMR spectrum of 2,4-dinitro-[¹³C₆]phenylhydrazine (300-MHz; solvent: CDCl₃; reference: Si(CH₃)₄).

According to the subject invention, where DNPH is labeled with ¹⁵N, a negative-ion atmospheric pressure chemical ion atmospheric pressure chemical ionization mass spectrum would show a +4 u shift in mass-to-charge values, when compared to the non-labeled standard DNPH (Aldrich, Milwaukee, Wis.). Where DNPH is labeled with ¹⁸O, negative-ion atmospheric pressure chemical ion atmospheric pressure chemical ionization mass spectrum would show a +4 u shift in mass-to-charge values, when compared to the non-labeled standard DNPH (Aldrich, Milwaukee, Wis.).

As described above, certain embodiments of the invention provide DNPH labeled with multiple isotopes. For example, in embodiments where DNPH is labeled with a combination of ¹⁵N and ¹³C₆, the negative-ion atmospheric pressure chemical ion atmospheric pressure chemical ionization mass spectrum would shows a +10 u shift in mass-to-charge values, when compared to the non-labeled standard DNPH (Aldrich, Milwaukee, Wis.).

### EXAMPLE 2

### Methodology for the Determination of Oxidatively Induced Carbonylation of Proteins

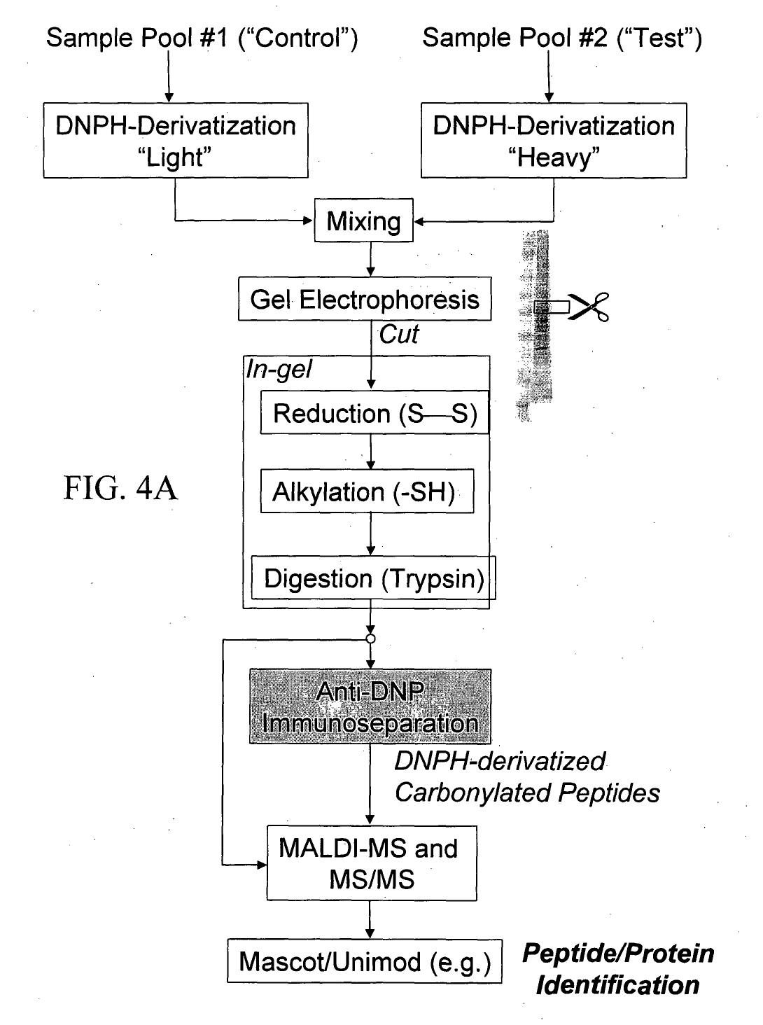

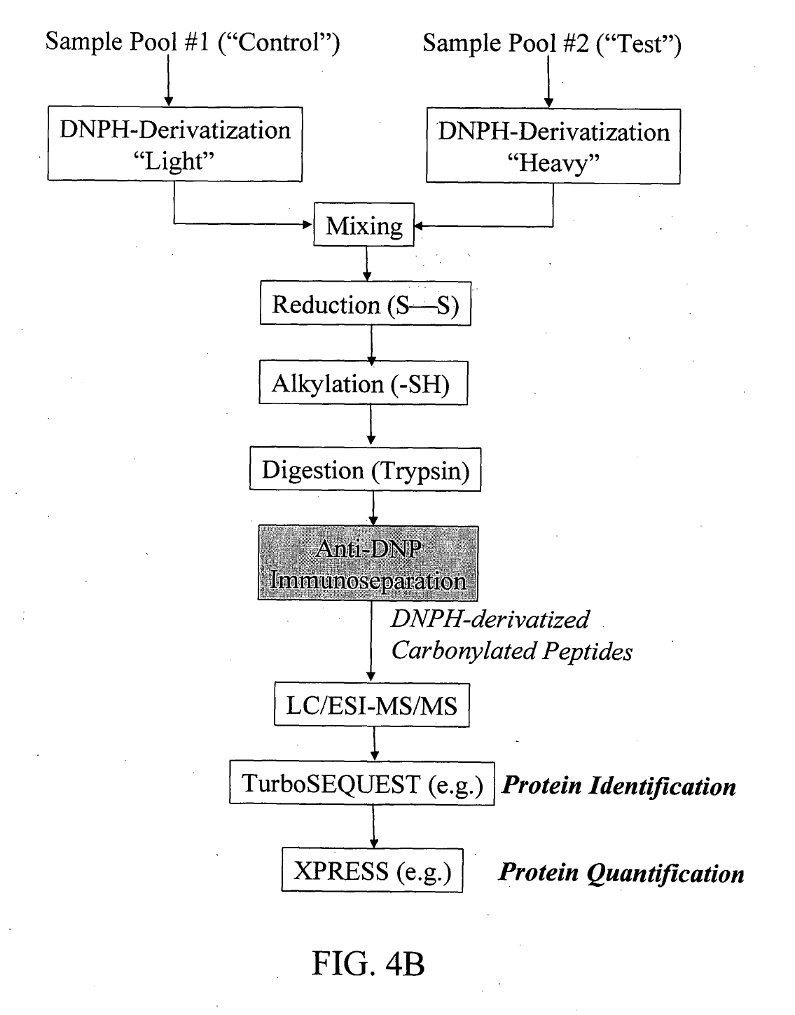

According to the subject invention, methods for detecting protein carbonylation can include a gel-based approach with mass spectrometry-based relative quantification, as illustrated in FIG. 4A. Other methods of the invention for detecting protein carbonylation include a gel-free approach based on LC/ESI-MS/MS, as illustrated in FIG. 4B. Schematic assay protocols and experimental details of the methods are given below.

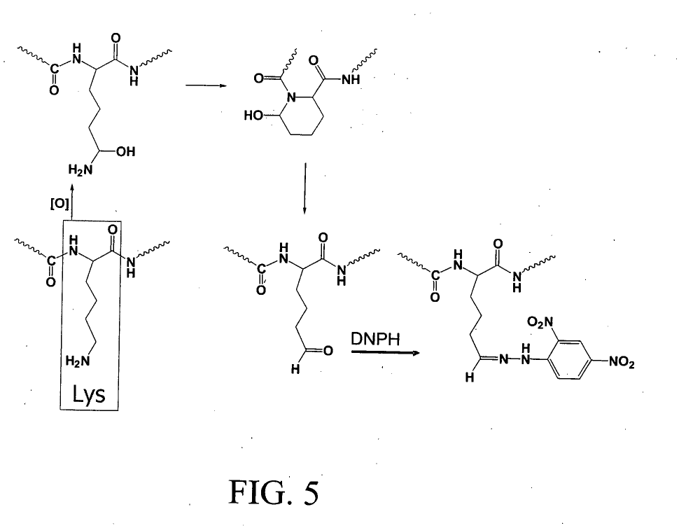

According to the subject invention, the first step in an ICAT-based strategy for detecting oxidatively induced carbonylation of proteins is protein derivatization by DNPH and stable-isotope labeled DNPH. Protein samples are derivatized in accordance with known protein derivatization methods (Levine, R. L. et al., “Determination of carbonyl content in oxidatively modified proteins,” Methods Enzymol., 186:464-478 (1990)). As an example, shown in FIG. 5, a lysine amino acid residue of a protein molecule undergoes oxidative conversion to a reactive carbonyl group. The reaction of the carbonyl with non-labeled DNPH (as well as isotope labeled DNPH) leads to the formation of a stable dinitrophenyl (DNP) hydrazone product. As understood by the skilled artisan, any protein amino acid residue having a hydroxyl group, such as lysyl, histidyl, arginyl, prolyl and threonyl residues, can undergo oxidative conversion to form carbonyl groups.

Examples of protein samples that can be used include: a control sample (such as Sample Pool #1 in FIGS. 4A and 4B), which can be a protein sample from a normally functioning organism or patient; and a test sample (such as Sample Pool #2 in FIGS. 4A and 4B), which can be a protein sample from a patient demonstrating an imbalance toward pro-oxidant activity of pro-oxidant/anti-oxidant homeostasis.

In accordance with one embodiment of the invention, protein samples are incubated with 10 mM unlabeled DNPH (also referred to herein as the “light” reagent”) and isotope labeled DNPH (also referred to herein as the “heavy” reagent) in 2N HCl (500 μL) for 1 h at room temperature in the dark, the reaction is then stopped and the proteins are precipitated by addition of trichloroacetic acid (TCA, 10% final concentration) and kept on ice for 10 min. Excess reagent may be removed by a series of ethanol:ethyl acetate resuspension/centrifugation steps. After derivatization, the sample pools to be compared are mixed together.

In certain embodiments, with two “heavy” reagents such as 2,4-dinitro-[¹³C₆]phenylhydrazine and 2,4-[¹⁵N¹⁸O₂]dinitrophyenylhydrazine that differ in mass from the unlabeled reagent by +6 u and +10 u, respectively, three samples can be mixed to compare two treatments to control and to each other simultaneously.

Gel-Based Approach with Mass Spectrometry-Based Relative Quantification

In one embodiment of the invention, as illustrated in FIG. 4A, after mixing together samples of derivatized protein, the next step is to undergo gel electrophoresis (such as sodium dodecyl sulfate-polyacrylamide gel electrophoresis or SDS-PAGE). Prior to ID SDS-PAGE separation, a 150 μl aliquot of the protein solution is combined with 150 μl of 2× Laemmli sample buffer (0.125 M Tris HCl, 4% SDS, 40% Glycerol, 0.1% Bromophenol blue, pH 6.8). Thirty microliters of 100 mM DTT is mixed with the solubilized protein and heated at 90° C. for 10 min. Fifteen microliters of the sample mixture (protein, Laemmli buffer, and DTT) is then loaded into each lane of a 15-lane, 4-20% gradient tris-glycine gel. For molecular weight calibration, 10 μl of SeeBlue Plus 2 protein standard mixture is added to the first lane of the well. After appropriate protein separation, the gel is stained overnight with 0.1% CBB R-250 (45% methanol, 10% acetic acid). The gel is then placed in 5% acetic acid/20% methanol where excess CBB that remained on the gel was removed. Bands with greater stain intensity and which correspond to proteins of molecular weights can be excised from the gel (generally, the width of the excision is ˜1 mm).

After gel electrophoresis, the next step, as illustrated in FIG. 4A is protein proteolysis (such as in-gel tryptic digestion). Selected bands or excised spots are digested in-gel with trypsin using a protocol similar to that described by the HHMI/Keck Facility at Yale University. Briefly, 1.5 ml Eppendorf tubes are prewashed with 500 μl 0.1% TFA/60% CH₃CN, the stained gel band (cut into small pieces) is put into a prewashed tube followed by the addition 250 μl 50% H₂O/50% acetonitrile. After washing for 5 min, the solution is removed and 250 μl of 50% CH₃CN/50 mM NH₄HCO₃ is added and the washing of the gel pieces is continued for an additional 30 min at room temp on a tilt table. The solution will then be removed and the gel pieces are dried completely by a centrifugal vacuum concentrator.

Subsequently, 0.1 μg modified trypsin (Promega) per 15 mm³ of gel in 15 μl 10 mM NH₄HCO₃ is added and let stand for 5-10 minutes to allow enzyme/buffer solution to absorb into the gel. An additional 20 μl 10 mM of NH₄HCO₃ that does not contain enzyme is then be added and the tube is incubated at 37° C. for 24 hours. The tryptic peptides are extracted (twice) for analysis by adding 200 μl 0.1% TFA, 60% CH₃CN and shaking at room temperature for 60 min. The combined extracts are dried by a centrifugal vacuum concentrator.

Prior to the MALDI-MS step as illustrated in FIG. 4A, the digested samples are concentrated onto a C18 ZipTip microcolumn, washed several times with 0.1% TFA, and eluted off the column onto the MALDI plate with 1 μL matrix solution. The digested sample is reconstituted in 90% acetonitrile/0.1% acetic acid, concentrated onto a HPL (hydrophilic absorbent) ZipTip microcolumn, washed with the reconstitution solution, and eluted off the microcolumn with 50% acetonitrile/0.1% acetic acid. The sample will then be centrifuged under vacuum until dryness and dissolved in 3% acetonitrile/0.5% acetic acid.

The matrix solution used for MALDI-MS may be prepared by dissolving 10 mg α-cyano-4-hydroxycinnamic acid in 1 mL of 60% acetonitrile/0.1% TFA. In addition to MALDI-MS, MS/MS can be performed (e.g., on a quadrupole/time-of-flight hybrid instrument) to obtain sequence tags for protein identification. Alternatively, LC/ESI-MS/MS analysis can (also) be performed. Mascot (Matrix Science, Inc.), Protein Prospector, SEQUEST (Thermo Electron Corp.) and UniMod can be used for protein database search and MS/MS data interpretation. Once the peptide/protein is identified, the intensity ratios obtained for the molecular ions of heavy/light ICAT-pairs in the MALDI-MS are used to calculate the relative quantity of the specifically carbonylated proteins in the sample pools compared.

Gel-Free Approach with LC/ESI-MS/MS Relative Quantification

In another embodiment of the invention, as illustrated in FIG. 4B, after mixing together samples of derivatized protein, the next step is to undergo reduction, alkylation and proteolysis (such as trypsinolysis) in solution. The DNPH-derivatized carbonylated protein is dissolved 100 μL of denaturing buffer (50 mM Tris, 0.1% SDS). After the addition of 5 μL of 50-mM dithiothreitol, the solution is heated at 60° C. for 20 minutes. Twenty-five microliters of 22-mM iodoacetamide solution is then added and the sample is incubated for 50 minutes at room temperature in the dark. After these reduction and alkylation steps, 5 μL of 0.1 μg/μL trypsin in 50 mM NH₄HCO₃ is added and the solution is incubated overnight at 37° C. Digested proteins from the gel are purified with ZipTip microcolumns as described above for the gel-based approach.

Following alkylation and proteolysis in solution, the digested sample is reconstituted in 90% acetonitrile/0.1% acetic acid, concentrated onto a HPL (hydrophilic absorbent) ZipTip microcolumn, washed with the reconstitution solution, and eluted off the microcolumn with 50% acetonitrile/0.1% acetic acid.

Then, as illustrated in FIG. 4B, the samples are subjected to LC/ESI-MS/MS analysis that employs a data-dependent acquisition strategy. Those peptide ions that exceed the threshold level (and heavy/light ICAT pairs with Δ=6/10, 3/5 and 2/3.33 u for singly-, doubly- or triply-charges molecular ions, depending on the mass for the “heavy” reagent, when the “mass-tag enabled” option is used) are then subjected to CID.

Protein identification from the collected data can be performed using, for example, TurboSEQUEST and an appropriate protein database, and the XPRESS tool (BioWorks 3.1, Thermo Electron Corp.) or another similar algorithm may be employed to obtain quantitative results from the ICAT experiments. For the submission of the data file to database search from the MS/MS of labeled and non-labeled DNPH-tagged tryptic fragments, the user can “custom-modify” the module accounting for post-translational modifications by defining the residues that are subject to carbonylation, give the mass difference carried by the modifications (including DNPH-derivatization), allow “dynamic” modification, and index the database for the proteolytic enzyme used (trypsin, allowing maximum of three missed cleavages). The list of matched peptides may be evaluated using the following criteria: (i) the presence of appropriate residue(s) in the peptide sequence for carbonylation, (ii) a satisfactory Xcorr value (Eng, J. K. et al., “An approach to correlate tandem mass spectral data of peptides with amino acid sequences in a protein database,” J Am Soc Mass Spectrom., 5:976-989 (1994)), e.g., >2.2 for doubly-charged ions and >3.5 for triply-charged ions, (iii) a delta correlation score of >0.3 and (iv) heavy/light ICAT-pairs exhibiting closely eluting peaks (scan numbers).

Additional Step of Derivatized Protein Contact with Anti-DNPH Immunosorbent

In certain embodiments, as illustrated in FIGS. 4A and 4B, after the derivatized proteins are proteolysed, an additional step of placing them in contact with anti-DNPH immunosorbent can be taken. To prepare anti-DNPH immunosorbent, in accordance with the subject invention, CNBr-activated sepharose 4B (1 g; Sigma, St. Louis, Mo.) is allowed to swell for about 15 min in 50 mL of 1 mM HCl. The resulting mixture is filtered and further rinsed twice with 50 mL of 1 mM HCl, then with 5 mL of a 0.1 M NaHCO₃ buffer (pH 8.3) containing 0.5 M NaCl (coupling buffer). From this swollen gel, an aliquot of 1 mL is shaken overnight at 4° C. with the anti-DNPH antibody (˜2 mg of protein; Molecular Probes, Eugene, Oreg.) in 5 mL of the coupling buffer.

After completion of the antibody-binding reaction, the sorbent is filtered and further treated with 5 mL of 0.1 M Tris-buffer (pH 8.0) containing 0.5 M of NaCl for 2 h at room temperature in order to block the excess of CNBr groups. The mixture obtained is then alternatively washed four times with 5 mL of coupling buffer and 5 mL of 0.1 M sodium acetate buffer pH 4.0 containing 0.5 M NaCl, to ensure that no free (non-covalently bound) ligand will remain adsorbed on the support. The anti-DNPH immunosorbent thus obtained is stored at 4° C. in 10 mM phosphate-buffered (pH 7.4) saline solution (PBS) containing 0.02% sodium azide.

Following protein proteolysis, the samples are subjected to DNP-antibody based immunoseparation using a DNPH immunosorbent slurry prepared as described above. The immunosorbent slurry (0.2 mL) is loaded into a 1-mL disposable Supelco (Bellefonte, Pa.) solid-phase extraction cartridge. The immunosorbent is conditioned with 3×0.8 mL of PBS and, then, with 3×0.8 mL of bidistilled water. Labeled and non-labeled DNPH-derivatized protein samples are dissolved in 4 mL of PBS and the subsequent solution (in 0.8-mL aliquots) is passed through the cartridge. After sample loading, the immunosorbent is washed twice with 3×0.8 mL of bidistilled water. The labeled and non-labeled DNPH-derivatized proteins will then be eluted with 0.8 mL of 0.1% TFA and the solution is evaporated to dryness in a centrifugal vacuum concentrator (Speed-Vac) into a 1.5-mL polypropylene centrifuge tube.

### EXAMPLE 3

### Methodology for the Determination of Oxidatively Induced Carbonylation of Plant Proteins

According to the subject invention, plant proteins implicated in oxidative stress can simultaneously and accurately identified and quantified using the methods described herein. Plant protein samples (both stressed (oxidatively) and unstressed samples) are derivatized in accordance with known protein derivatization methods (Kristensen, B. et al., “Identification of oxidized proteins in the matrix of rice leaf mitochondria by immunoprecipitation and two-dimensional liquid chromatography-tandem mass spectrometry,” Phytochemistry, 65:1839-1851 (2004)).

For example, plant leaves can be homogenized in a variety of known ways to test whether the procedure introduces protein oxidation (for example, as known to the skilled artisan, homogenization can be performed with either with an UltraTurrax (IKA Werke, Germany), a Warring blender, or grinding in a mortar). Pure mitochondria (mitochondria matrix protein) is then extracted from the homogenate using known techniques (such as centrifuge, resuspension, etc.).

To subject the samples to oxidative stress, the mitochondrial matrix protein (2.0 mg) is treated with metal-catalysed oxidation reagent for 10 min at room temperature (22-23° C.). In the control sample (2.0 mg), the metal-catalysed oxidation reagent was omitted. Adding EDTA, pH 7.0 to a final concentration of 10 mM and freezing in liquid nitrogen stopped the reaction.

In accordance with one embodiment of the invention, protein samples are incubated with 10 mM unlabeled DNPH (also referred to herein as the “light” reagent”) and isotope labeled DNPH (also referred to herein as the “heavy” reagent) in 2N HCl (500 μL) for 1 h at room temperature in the dark, the reaction is then stopped and the proteins are precipitated by addition of trichloroacetic acid (TCA, 10% final concentration) and kept on ice for 10 min. Excess reagent may be removed by a series of ethanol:ethyl acetate resuspension/centrifugation steps. After derivatization, the sample pools to be compared are mixed together.

In certain embodiments, with two “heavy” reagents such as 2,4-dinitro-[¹³C₆]phenylhydrazine and 2,4-[¹⁵N¹⁸O₂]dinitrophyenylhydrazine that differ in mass from the unlabeled reagent by +6 u and +10 u, respectively, three samples can be mixed to compare two treatments to control and to each other simultaneously. Schematic assay protocols and experimental details for simultaneous relative quantification of oxidized proteins are given above in Example 2 (see gel-based and gel-free approaches described above).

In another aspect, the functionality of one or more of the methods described above may be implemented as computer-readable instructions on a general purpose computer. The computer may be separate from, detachable from, or integrated into a mass spectrometry system. The computer-readable instructions may be written in any one of a number of high-level languages, such as, for example, FORTRAN, PASCAL, C, C++, or BASIC. Further, the computer-readable instructions may be written in a script, macro, or functionality embedded in commercially available software, such as EXCEL or VISUAL BASIC. Additionally, the computer-readable instructions could be implemented in an assembly language directed to a microprocessor resident on a computer. For example, the computer-readable instructions could be implemented in Intel 80x86 assembly language if it were configured to run on an IBM PC or PC clone. In one embodiment, the computer-readable instructions can be embedded on an article of manufacture including, but not limited to, a computer-readable program medium such as, for example, a floppy disk, a hard disk, an optical disk, a magnetic tape, a PROM, an EPROM, or CD-ROM.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety to the extent they are not inconsistent with the explicit teachings of this specification.

1. A method for identifying and/or quantifying analytes susceptible to oxidation comprising:
(a) reacting a first sample of analytes with DNPH to produce a first sample of DNPH-treated analytes;
(b) reacting a second sample of analytes with an isotopically-labeled DNPH to produce a second sample of iso-DNPH-treated analytes;
(c) combining the samples of DNPH-treated and iso-DNPH-treated analytes;
(d) subjecting the DNPH-treated and iso-DNPH-treated analytes to separation and proteolysis; and
(e) determining the identity and/or quantity of analytes susceptible to oxidation from the separated and proteolysed DNPH-treated and iso-DNPH-treated analytes.

2. The method of claim 1, wherein the analytes are selected from the group consisting of: proteins, lipids, DNA, RNA, glycoproteins; nucleic acids; neurotransmitters; hormones; growth factors; antineoplastic agents; cytokines; monokines; lymphokines; enzymes; receptors; animal and plant cells; stem cells; and blood cells; microorganisms; fungi; viruses; yeast; mycoplasms; gram positive and gram negative bacteria; protozoa; and any combination thereof.

3. The method of claim 1, wherein the isotopically-labeled DNPH is selected from the group consisting of: 2,4-dinitro-[¹³C₆]phenylhydrazine; 2,4-di-[¹⁵N]nitrophenylhydrazine; 2,4-di-[¹⁸O]nitrophenylhydrazine; 2,4-dinitrophyenyl[¹⁵N]hydrazine; 2,4-di-[¹⁵N,¹⁸O]nitro-phenyl-hydrazine; and 2,4-di-[¹⁵N, ¹⁸O]nitrophyenyl[¹⁵N]hydrazine.

4. The method of claim 1, wherein the DNPH-treated and iso-DNPH-treated analytes are separated using either gel-based or gel-free separation methods.

5. The method of claim 4, wherein the gel-based separation method is one-dimensional or two-dimensional gel electrophoresis.

6. The method of claim 1, further comprising the step of contacting the separated and/or proteolysed DNPH-treated and iso-DNPH-treated analytes with anti-DNPH immunosorbent prior to the step of determining the specifically carbonylated analytes.

7. The method of claim 1, wherein determination of quantity and/or identity of analytes susceptible to oxidation is accomplished using any one or combination of the techniques selected from the group consisting of: spectrophotometric assay; enzyme-linked immunosorbent assay (ELISA); one-dimensional or two-dimensional electrophoresis followed by Western blot immunoassay; liquid chromatography (LC); two-dimensional liquid chromatography (2D-LC); mass spectrometry (MS); matrix assisted laser desorption/ionization mass spectrometry (MALDI-MS); high-performance liquid chromatography (HPLC); HPLC-mass spectrometry (HPLC-MS); electrospray ionization (ESI); atmospheric pressure chemical ionization (APCI); and selected ion monitoring (SIM) and/or selected reaction monitoring (SRM).

8. The method of claim 7, wherein determination of specifically carbonylated analytes is accomplished using any one or combination of the techniques selected from the group consisting of: LC/MS; HPLC; LC/ESI-MS/MS; MALDI-MS; and MS/MS.

9. The method of claim 1, further comprising the step of identifying a disease, disorder, or condition associated with the analytes susceptible to oxidation as determined from step (e).

10. The method of claim 9, wherein the disease, disorder, or condition is selected from the group consisting of: age-associated dementia; Alzheimer's disease; Parkinson's disease; amyotrophic lateral sclerosis (ALS); multiple sclerosis; peripheral neuropathy; shingles; stroke; traumatic injury; schizophrenia; epilepsy; Down's Syndrome; Turner's Syndrome; degenerative conditions associated with AIDS; osteoporosis; osteomyelitis; ischemic bone disease; fibrous dysplasia; rickets; Cushing's syndrome; osteoarthritis; rheumatoid arthritis; psoriatic arthritis; infectious arthritis; infectious diseases; muscular dystrophy; dermatitis; eczema; psoriasis; skin aging; degenerative disorders of the eye; macular degeneration; retinal degeneration; disorders of the ear; otosclerosis; impaired wound healing; cardiovascular diseases; cardiovascular conditions; stroke; cardiac ischemia; myocardial infarction; chronic heart failure; heart failure; cardiac dysrhymias; artrial fibrillation; paroxysmal tachycardia; ventricular fibrillation; congestive heart failure; circulatory disorders; atherosclerosis; arterial sclerosis; peripheral vascular disease; diabetes; lung disease; lung cancer; pneumonia; chronic obstructive lung disease; bronchitis; emphysemia; asthma; disorders of the gastrointestinal tract; ulcers; hernia; dental conditions; periodontitis; liver disease; hepatitis; cirrhosis; pancreatic ailments' acute pancreatitis; kidney disease; acute renal failure; glomerulonepritis; blood disorders; vascular amyloidosis; aneurysms; anemia; hemorrhage; sickle cell anemia; autoimmune disease; red blood cell fragmentation syndrome; neutropenia; leucopenia; bone marrow aphasia; pancytopenia; thrombocytopenia; and hemophilia.

11. The method of claim 1, wherein the analyte susceptible to oxidation as determined from step (e) is a protein from plant mitochondria.

12. A compound for identifying and/or quantifying analytes susceptible to oxidation comprising an isotopically-labeled DNPH.

13. The compound of claim 12, wherein the isotopically-labeled DNPH is labeled with ¹³C, N, ¹⁸O, or ²H.

14. A composition for identifying and/or quantifying analytes susceptible to oxidation comprising an isotopically-labeled DNPH.

15. The composition of claim 14, wherein the isotopically-labeled DNPH is labeled with ¹³C, ¹⁵N, ¹⁸O, or ²H.
